(19) United States
(12) Patent Application Publication
(10) Pub. No.: US 2017/0335263 A1
(43) Pub. Date: Nov. 23, 2017

(54) CONTAINER FOR RAISING AND STORING LIQUIDS SUCH AS WINE

(71) Applicant: RTECH SOLUTIONS, Levernois (FR)

(72) Inventor: Arnaud ROUSSAC, Beaune (FR)

(73) Assignee: RTECH SOLUTIONS, Levernois (FR)

(21) Appl. No.: 15/532,580

(22) PCT Filed: Dec. 2, 2015
(86) PCT No.: PCT/FR2015/053302

§ 371 Date: Jun. 2, 2017

(30) Foreign Application Priority Data
Dec. 2, 2014 (FR) 14 61758

(51) Int. Cl. C12H 1/16; C12H 1/22; A61L 2/10; B08B 9/08; A61L 2/07; C12L 9/00; B65D 8/00

(52) U.S. Cl. CPC C12H 1/16; C12L 9/00; C12H 1/22; B65D 15/10; B08B 9/0813; A61L 2/07; B08B 9/0808; A61L 2/10; A61L 2202/17; A61L 2202/23

(57) ABSTRACT

A container for raising and storing wine, which includes an external metal enclosure having at least one body in the overall shape of a hollow cylinder and a rear wall fixed to the longitudinal rear end of the body in order to close it off, an internal wooden enclosure that is able to contain the wine, including at least one body in the overall shape of a hollow cylinder and a front wall and a rear wall that close off the body in a leaktight manner, the internal enclosure being inserted into the external enclosure with a tight fit until the rear wall of the internal enclosure butts against the rear wall of the external enclosure, and clamping elements that exert a longitudinal force that tends to clamp the internal enclosure against the external enclosure.

### TECHNICAL DOMAIN

This invention relates to the domain of liquid containers, and more specifically, a container for raising and storing wine. This invention also relates to a method for cleaning and sterilising said container, in view of its storage when empty.

### PRIOR TECHNIQUE

It is well known to use wooden barrels to raise and keep wine. These barrels are usually constructed from wooden oak staves, curved when hot and held together by external strapping, the oak wood being able to supply desirable characteristics during the formation of wine. Indeed, during the ageing process, the wine acquires a suitable amount of oxygen and tannin passing through the oak pores, in particular to achieve the desired oak and tannin aroma.

Consequently, as time passes, the quantity of tannin received by the wine from the wood pores inside the barrels decreases in a damaging way, until generally needing the oak barrels to be replaced.

Traditional barrels, which classically have a content of 225 litres, are generally used as a maximum, or 3 times before a replacement proves to be necessary. For winemakers, replacing all or part of their barrel stock represents a very significant investment.

Indeed, other than the increased cost of manufacturing barrels, due in part to the specific labour required, in particular, manufacturing and curving staves, oak wood is becoming rare and therefore very expensive.

Moreover, traditional barrels require a ventilated storage space, as wine needs oxygen to be raised, of which the temperature must be regulated, as the temperature inside the barrels is dependent on the room temperature of said storage space.

### DISCLOSURE OF THE INVENTION

The aim of this invention is therefore to alleviate the disadvantages previously cited and to propose a container for raising and storing wine, requiring, at an equal volume, a quantity of oak wood, reduced compared with a traditional barrel, while ensuring the exchanges between the wine and the wood, necessary for giving it the aroma and for the raising of said wine stored in said container. Additionally, the container, according to the invention, enables to not have to curve the staves, avoiding having to resort to specialist labour, while guaranteeing the correct mechanical strength of said container.

Conforming with the invention, a container for raising and storing wine is therefore proposed, noteworthy in that it comprises an external metal enclosure comprising at least one body in the overall shape of a hollow cylinder and a rear wall fixed to the longitudinal rear end of said body in order to close it off, an internal wooden enclosure, that is able to contain the wine comprising at least one body in the overall shape of a hollow cylinder and a front wall and a rear wall, that close off said body in a leaktight manner, said internal enclosure being inserted into said external enclosure with a tight fit until said rear wall of the internal enclosure butts against the rear wall of said external enclosure, and clamping elements that exert a longitudinal force that tend to clamp said internal enclosure against the rear wall of said external enclosure.

It is understood that the invention enables the working of the wood to be facilitated and to significantly reduce the quantity of wood necessary, as the internal wooden enclosure containing the wine only fulfils the function of giving an aroma, all mechanical strength being regained by the external metal enclosure.

The external enclosure is preferably made of stainless steel.

The internal enclosure is advantageously made from a mix of wood species and a type of heat from said species.

Advantageously, said front and rear walls respectively comprise a peripheral groove for enable the respective interlocking of the peripheral front and rear edges of said body.

According to a preferred embodiment, the body is formed from staves that have a straight-lined neutral fibre and a cross-section in the overall shape of an isosceles trapezium; said staves being positioned in a way to put one of their tiled longitudinal faces in contact with another face, while taking care to place their small base inside said body.

The container according to the invention advantageously comprises a leaktight seal, compatible with the wine, in the spaces between, on the one hand, said peripheral grooves and front and rear peripheral edges of said body and, on the other hand, the tiled longitudinal faces.

According to a preferred embodiment, the body of the external enclosure has a polygonal shape, of which the inside cross-section is homothetic and of very slightly larger dimensions that those of the external cross-section of the body of the internal enclosure.

Advantageously, the container comprises an automatic wine-producing system.

Said automatic wine-producing system comprises at least:
one wine oxygenating device,
one independent thermoregulating device,
one pressure control device in the internal enclosure, and
one graphic management interface, enabling all devices of the automatic wine-producing system to be managed, and to quickly and simply control the progression of the wine's fermentation state.

Said oxygenating device preferably comprises an oxygen consumption measuring body, a wine oxygen saturation control body and an oxygen inflow body.

Said independent thermoregulating device comprises a wine temperature measuring probe and a heat exchanger, connected to a network of pipes, positioned over the whole of the external periphery of the body of the external enclosure, said pipes heating or cooling through conduction of said external enclosure.

Said automatic wine-producing system can also comprise a brewing device and a sediment-extracting device.

The invention also concerns a method of cleaning and sterilising the container according to the invention in view of its storage when empty, comprising the following steps:
brushing the whole of the inside surface of the internal enclosure,
cleaning the whole of the inside surface of the internal enclosure with a high-pressure water jet,
deep-sterilising the inside of the internal enclosure with steam or with ultraviolet rays.

### SUMMARISING DESCRIPTION OF THE FIGURES

Other benefits and characteristics will emerge better from the description which will follow a variant of the embodiment of a wine-making container for raising and storing wine, according to the invention referenced in the appended figures, whereon:

FIG. 1 is a perspective view of a wine-making container, for raising and storing wine according to the invention;

FIG. 2 is a longitudinal cross-sectional view of the container of FIG. 1 according to the axis II-II′ of FIG. 1;

FIG. 3 is a perspective view of the internal enclosure of the container of FIG. 1;

FIG. 4 is a longitudinal cross-sectional view of the internal enclosure of FIG. 3 according to the axis IV-IV′ of FIG. 3;

FIG. 5 is a straight cross-sectional partially detailed view of the container of FIG. 1;

FIGS. 6 to 8 are perspective views showing the different steps of implementing the internal enclosure of FIG. 3;

FIG. 9 is a straight cross-sectional view of a variant of the embodiment of the wine-making container, for raising and storing wine according to the invention;

FIG. 10 is a straight cross-sectional view of a stave of the container of FIG. 9.

### BEST WAY TO ACHIEVE THE TECHNICAL INVENTION

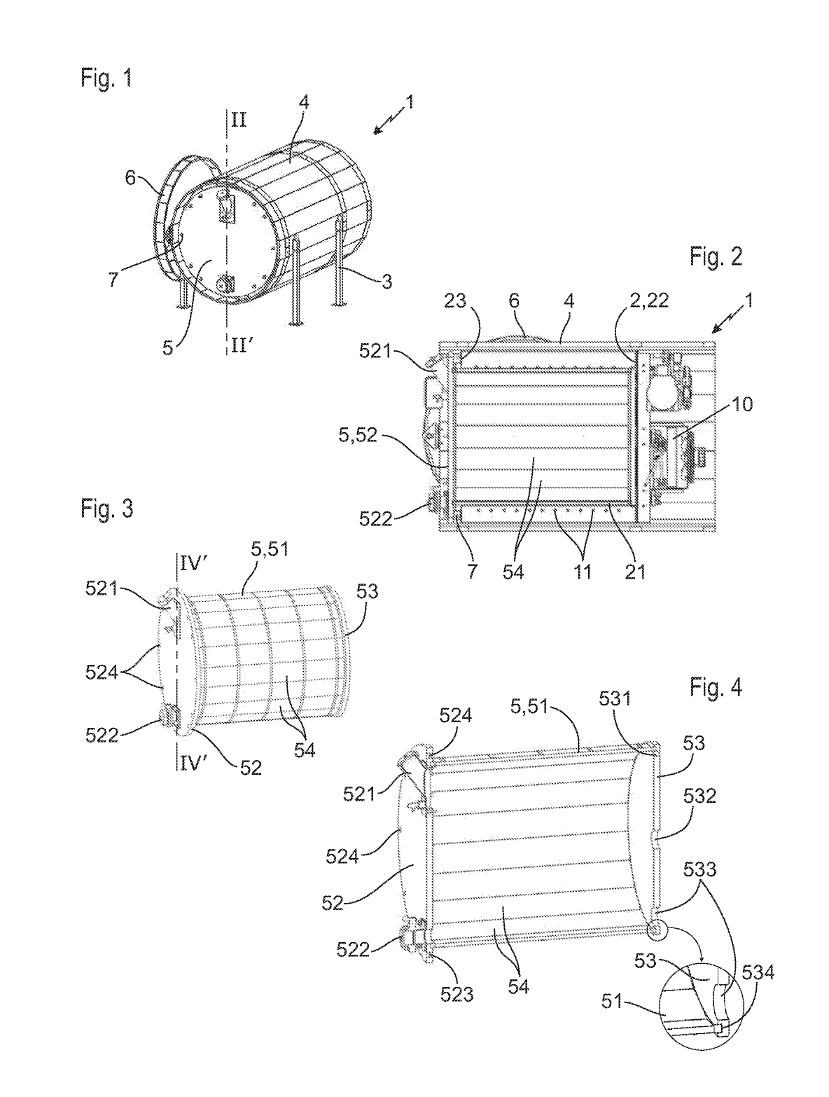

In reference to FIGS. 1 and 2, the wine-producing container 1, for raising and storing liquid, such as preferably wine, according to the invention, comprises an external enclosure 2 equipped with feet 3 disposed to rest on the floor of a wine storehouse, for example, and advantageously covered with a plurality of cladded boards 4, an internal enclosure 5 inserted inside said external enclosure 2 with a tight fit, and a removable cladded front 6, able to close the external enclosure 2 to conceal the internal enclosure 5.

The external enclosure 2 comprises at least one body 21 in the overall shape of a hollow cylinder, a rear wall 22 fixed on the longitudinal rear end of said body 21 to close it off, and a front flange 23 fixed around the longitudinal front end of said body 21. For mechanical strength and corrosion resistance reasons, the external enclosure 2 is metal and made preferably from stainless steel.

It is reminded that a cylinder (or cylinder shape) is a surface in the space defined by a straight section, called generating, passing through a variable point describing a closed flat curve, called directing curve and keeping a fixed direction.

Here, “front” is meant by the element, such as, for example, wall or face which is located on the user's side when the container 1 according to the invention is used under normal conditions, namely installed in a wine storehouse for example, and by “rear”, the element located on the opposite side of said front element.

According to a preferred embodiment, the cross-section of the body 21 is a polygonal section.

The internal enclosure 5, which is intended to contain the wine, is made from wood, in order to guarantee an aroma to said wine. The internal enclosure 5 comprises a body 51 in the overall shape of a hollow cylinder, a front wall 52 and a rear wall 53, said front and rear walls 52, 53 being able to conceal the longitudinal ends of said body 51 in a leaktight manner. This body is made from straight-lined staves 54.

It is known that wine is a complex element, of which the taste is highly influenced by the wood species used to manufacture the container and the type of heat applied to said wood. That is why, wood is used to make the internal enclosure 5.

However, it is essential to know and to understand the different types of heat which will all be beneficial with a radiation heat system, enabling the distribution of temperature and the heated depth of the wood to be controlled, in order to guarantee consistent and repeated heat.

Thus, the wood used to make the internal enclosure 5 can undergo one of the following types of heat:
Low heat, guaranteeing a discreet fruity aroma;
Average heat, guaranteeing complex aromas: lightly toasted bread aroma, mixing with fruity and cinnamon aromas, the average heat enabling the tannins to integrate into the original structure of the wine;
Above-average heat, guaranteeing very complex aromas: blend of caramel, low roasting and spicy aromas, the tannins thus being thermally degraded and coating the wine tannins;
High heat, guaranteeing powerful aromas: blend of roasting aromas, bitter chocolate and cocoa, the tannins thus being highly thermally degraded, which has the effect of bringing hardly any tannins rounding those of the wine.

The configuration of the internal enclosure 5 enables a mix of different types of heat. Thus, in one same internal enclosure 5, for example, there can be staves 54 heated according to the heat type “low heat”, and other heats according to the heat type “high heat”.

Likewise, the configuration of the internal enclosure 5 enables a mix of different types of wood species. Thus, in one same internal enclosure 5, for example, there can be staves 54 made from one first wood species and others made from a first wood species, the wood species being preferably chosen from the group of following traditional wood species: oak, acacia, chestnut, ash, pine, locust, fir, spruce, Douglas fir, maritime pine, Scotch pine, poplar, beech, plane, aspen, alder, olive, birch.

Thus, by mixing the wood species and the heat type for one same internal enclosure 5, in particular regarding its staves 54, a complex aromatic palette enabling said internal enclosure 5 to be adapted to the aromatic needs of the wine.

In reference to FIGS. 3 and 4, the front wall 52 of the internal enclosure 5, which is a disc in an overall circular shape, having a diameter larger than the width outside of the whole body 51, ensures it is leaktight with said body 51 formed by the staves 54 and also enables different bodies to be fixed, necessary for the different stages of wine-producing.

For this, this front wall 52 comprises at least:
one bung 521 coming from the upper median area of the front face of said front wall 52 and extending towards the top and the front of the internal enclosure 5,
one emptying valve 522 coming substantially perpendicularly from the lower median area of the front face of said front wall 52 and extending towards the front of the internal enclosure 5,
one peripheral groove 523 positioned on the rear face of said front wall 52 to enable the interlocking of the peripheral front edge of the body 51, and
multiple cross-section orifices 524, advantageously spread out regularly along a diameter larger than the width outside of the whole of the body 51.

Said bung 55 and valve 56, respectively enabling the filling and emptying of the internal enclosure 5.

In reference to FIGS. 3 and 4, the rear wall 53 of the internal enclosure 5, which is a disc in an overall circular shape, having a diameter smaller than or equal to the width outside of the whole body 51, ensures it is leaktight with said body 51 formed by the staves 54 and also enables different bodies to be fixed, necessary for the different stages of wine-producing.

For this, according to a preferred embodiment, this rear wall 53 comprises at least:
a first orifice 531 crossing positioned in the upper median area of the front face of said rear wall 53,
a second orifice 532 crossing positioned in the median area of the front face of said rear wall 53,
a third orifice 533 crossing positioned in the lower median area of the front face of said rear wall 53,
a peripheral groove 534 positioned on the front face of said rear wall 53 and enabling the interlocking of the rear peripheral edge of the body 51.

Said first, second and third orifices 531, 532, 533 are positioned in the area of the rear wall 53, located inside the groove 534.

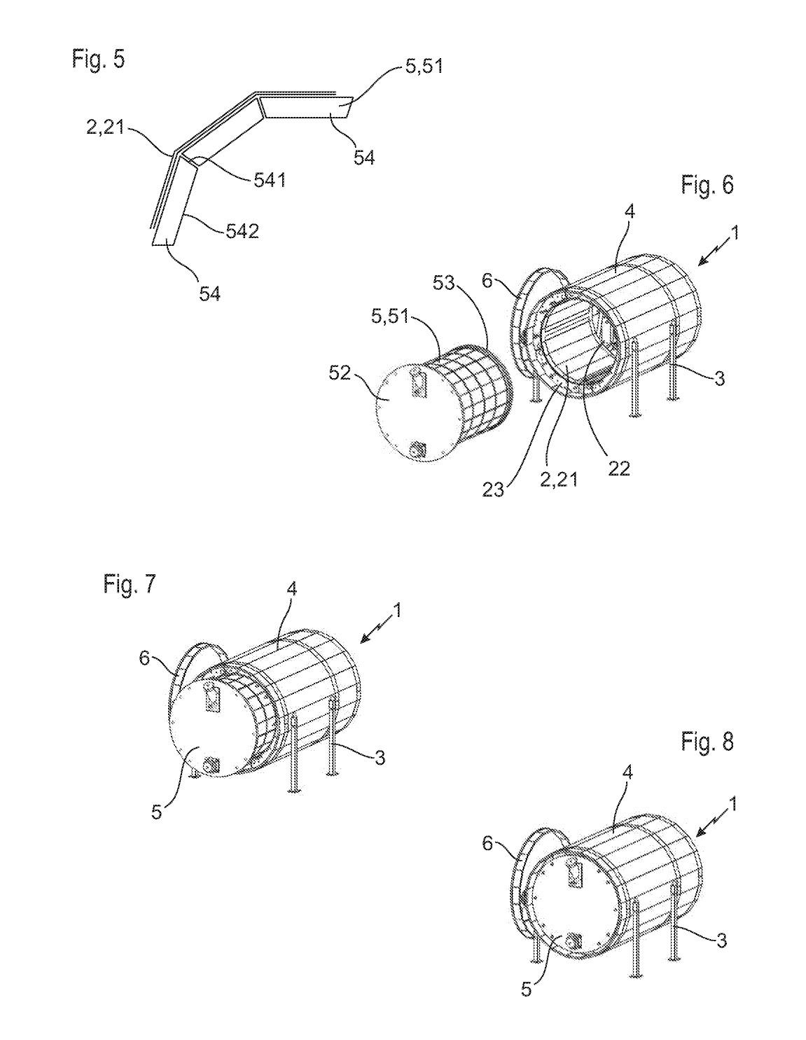

The staves 54 have a straight-lined neutral fibre and a cross-section in the overall shape of an isosceles trapezium. To form the internal enclosure 5, all that is needed therefore, in reference to FIG. 5, is to shape the body 51 by positioning the adjacent staves 54 in a way for them to be in contact with one of their tilted longitudinal faces 541, while taking care to place their small base 542 inside said body 51. Once the body 51 is formed, the front and rear peripheral edges of said body 51 need to be interlocked respectively in the peripheral groove 523 of the front wall 52 and in the peripheral groove 534 of the rear wall 53.

To guarantee that the internal enclosure 5 is perfectly leaktight, advantageously there are in the spaces between, on the one hand, said peripheral grooves 523, 534 and the front and rear peripheral edges of said body 51 and, on the other hand, the tilted longitudinal faces 541, a leaktight seal compatible with wine, this seal being, for example, a starch-based seal.

In reference to FIGS. 6 to 8, once the internal enclosure 5 is constituted, it is inserted by its rear wall 53 into the external enclosure 2 until when this rear wall 53 butts against the rear wall 22 of said external enclosure 2 (see FIG. 2).

To ensure the internal enclosure 5 is maintained, the external enclosure 2 additionally comprises clamping elements 7, for example, nut and dowel sets, coming substantially perpendicularly from the front face of its flange 2 and spread out regularly along a diameter, said clamping elements 7 being able to cooperate with the orifices 524 of the front wall 52 of the internal enclosure 5, in order to exert a longitudinal force that tends to clamp said internal enclosure 5 against the rear wall 22 of said external enclosure 2 and therefore clamp together the set of elements constituting said internal enclosure 5.

The external enclosure 2 has a polygonal shape, of which the internal cross-section is homothetic and has very slightly larger dimensions to that of the external cross-section of the body 51 of the internal enclosure 5 in order to enable an insertion with a tight fit of the internal enclosure 5 into the external enclosure 2, in other words, that once the insertion is made, there is contact between the internal enclosure 5 and the external enclosure 2, without the possibility for related cross-sectional movements of the internal enclosure 5 in relation to the external enclosure 2.

A person skilled in the art will have no difficulty in determining the value of tolerances to implement to achieve an insertion of the internal enclosure 5 into the external enclosure 2 with a tight fit.

Thus, the external enclosure 2 contributes to keeping the staves 54 in contact with each other, and regains the mechanical strength subjected to by the internal enclosure 5, due in particular, to the increase in pressure in this internal enclosure during the alcoholic fermentation of the wine. This configuration enables the thickness of the staves 54 to be reduced, and therefore to significantly reduce the cost of the internal enclosure 5.

Moreover, with this configuration of the container 1 according to the invention, to renew the internal enclosure 5 with wood after 2 or 3 uses, all is needed, is to disassemble the front wall 52, then one of the staves 54 acting as a “cornerstone”, the others subsequently falling all by themselves.

It is well understood that this configuration of the container 1 according to the invention enables the elements constituting the container to be partially renewed, in order to highly reduce its cost and to preserve natural wood resources by reducing its need for wood.

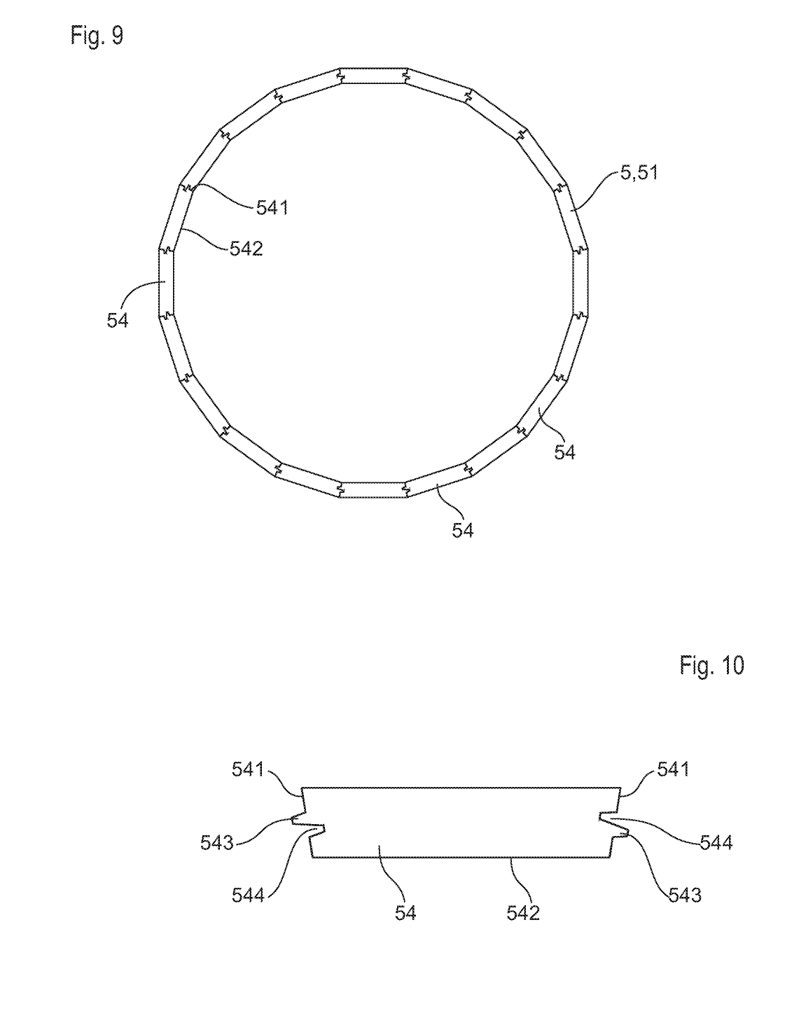

According to a variant of the embodiment represented in FIGS. 9 and 10, the staves 54 have a straight-lined neutral fibre and a cross-section in the overall shape of an isosceles trapezium. To shape the internal enclosure 5, all is needed therefore, in reference to FIG. 5, is to shape the body 51 of said internal enclosure 5 by positioning the adjacent staves 54 in a way for them to be in contact with one of their tilted longitudinal faces 541, while taking care to place their small base 542 inside said body 51.

However, to increase the mechanical resistance of the body 51 subsequently shaped, each stave 54 comprises at least one longitudinal pin 543 and one longitudinal groove 544 disposed along each one of its tilted longitudinal faces 541 (see FIG. 10), said longitudinal pin 543 and longitudinal groove 544 being able to cooperate respectively with the longitudinal groove 544 and longitudinal pin 543 of the adjacent stave 54 (see FIG. 9).

According to a preferred embodiment, the container 1 according to the invention also comprises an automatic wine-producing system 10 in the container 1 and preferably positioned behind the rear wall 22 of the external enclosure 2 to enable, in particular, the temperature, the pressure, the quantity of carbon dioxide and the oxygen inflow to be controlled during the alcoholic fermentation.

For this, this external enclosure comprises first, second and third orifices, not represented, similar to the first, second and third orifices 531, 532, 533 of the rear wall 53 of the internal enclosure 5 and set up on its rear wall 22 to the right of said first, second and third orifices 531, 532, 533. These different orifices enable equipment from the automatic wine-producing system 10 to pass through.

The container 1 additionally comprises leaktight bodies, not represented, to ensure a leaktight connection between the first, second and third orifices of the rear wall 22 and the first, second and third orifices 531, 532, 533 of the rear wall 53 at the time of insertion of the internal enclosure 5 into the external enclosure 2.

The automatic wine-producing system 10 comprises at least the following non-represented devices:
one wine oxygenation device,
one independent thermoregulation device,
one pressure control device in the internal enclosure 5, and
one graphic management interface enabling all devices to be managed from the automatic wine-producing system 10 and to quickly and simply control the progression of the fermentation state of the wine.

The oxygenation device ensuring a slow and regular inflow of oxygen, adapted to each type of wine. Indeed, it is important for the quality of the wine to control its concentration and its speed in consuming oxygen, as wine in contact with oxygen absorbs it continually until saturation. Without control, there are risks of oxidation, dry tannins, unstable colours and an absence of tannic development.

To do this, said oxygenation device comprises a body for measuring oxygen consumption, a body for controlling oxygen saturation of the wine and a body for oxygen inflow crossing, preferably, the third respective orifices 533 of the rear walls 22, 53 of the external enclosure 2 and internal enclosure 5, leaktight. The bodies for measuring oxygen consumption and controlling oxygen saturation of the wine cross in a leaktight manner, preferably, the first respective orifices 531 of the rear walls 22, 53 of the external enclosure 2 and internal enclosure 5.

The independent thermoregulation device ensures a consistent wine temperature, independent from the room temperature of the wine storehouse or the cellar.

To do this, said independent thermoregulation device comprising a probe for measuring the temperature of the wine and a thermal exchanger connected to a network of pipes 11 positioned over the whole of the external periphery of the body 21 of the external enclosure 2, said pipes 11 heating or cooling said external enclosure 2 through conduction, and therefore also the internal enclosure 5 of the wine because of the contact between the internal enclosure 5 and the external enclosure 2.

To encourage thermal exchanges between the pipes 11 and the external enclosure 2 and avoid wastage to the outside of the container 1, this container comprises a layer of insulation 1 between said pipes 11 and cladded boards 4.

According to a beneficial embodiment, the automatic wine-producing system 10 also comprises a brewing device and a sediment-extracting device, not represented.

The brewing device, which enables to wine contained in the internal enclosure 5 to be standardised, comprises a rotating shaft connected to an actuator and a brewer interdependent from said rotating shaft, this rotating shaft preferably passing through the second respective orifices 532 of the rear walls 22, 53 of the external enclosure 2 and internal enclosure 5.

The sediment-extracting device, which enables sediment collected from the lower section of said internal enclosure 5 to be extracted from the internal enclosure 5, comprises a collector in the form of a hopper and a bleed screw moved by an actuator and of which the rotation drives the sediment towards the outside of the internal enclosure 5, preferably through the third respective orifices 533 of the rear walls 22, 53 of the external enclosure 2 and internal enclosure 5.

It is well understood that the container 1 according to the invention, keeps all the benefits from raising in the wooden barrel (woody aromas, oxygen inflow), by controlling the oxygenation and by guaranteeing a consistent and stable temperature of the wine all throughout the raising. Likewise, the container 1 optimises the start-up conditions and maintains malolactic fermentation, whatever the temperature of the cellar or the wine storehouse.

This invention also concerns a method of cleaning and sterilising the container 1 according to the invention, in view of its storage when empty.

Said cleaning and sterilising method comprises at least, preferably, the following steps:
brushing the whole of the inside surface of the internal enclosure 5,
cleaning the whole of the inside surface of the internal enclosure 5 with a high-pressure water jet,
deep-sterilising the inside of the internal enclosure 5 with steam or with ultraviolet rays.

It is understood that these steps cannot be executed in the order indicated. For example, the brushing can be carried out after high-pressure cleaning, even at the same time.

The two first steps require the internal enclosure 5 to be opened and the brushing and cleaning of the front wall 52 and the whole of the body 51 and rear wall 53 separately, while the third step is carried out on the closed internal enclosure 5, and the steam generator or ultraviolet rays are introduced by the bung 521 of the front wall 52.

### Possibility for Industrial Application

It is well understood that the container 1 according to the invention is particularly suitable for raising and storing wine. However, this container 1 can also be applied to any other liquid, such as whisky for example.

Finally, it goes without saying that the example of container 1 that conforms to the invention which has just been described is only a specific illustration, in no case limiting the invention.

1-13. (canceled)

14. Container for raising and storing wine, comprising:
an external metal enclosure, comprising at least one body in the overall shape of a hollow cylinder and one rear wall fixed to the longitudinal rear end of said body to close it off, an internal wooden enclosure, that is able to contain the wine, comprising at least one body in the overall shape of a hollow cylinder and a front wall and rear wall that closes off said body in a leaktight manner, said internal enclosure being inserted into said external enclosure, with a tight fit until said rear wall of the internal enclosure butts against the rear wall of said external enclosure, and clamping elements that exert a longitudinal force that tends to clamp said internal enclosure against the rear wall of said external enclosure.

15. Container according to claim 14, wherein the external enclosure is made from stainless steel.

16. Container according to claim 14, wherein the internal enclosure is made from a mix of wood species and type of heating said species.

17. Container according to claim 14, wherein said front wall and rear wall respectively comprise a peripheral groove to enable the respective interlocking of the front and rear peripheral edges of said body.

18. Container according to claim 14, wherein said body is shaped from staves that have a straight-lined neutral fibre and a cross-section in the overall shape of an isosceles trapezium, said staves being positioned in a way to contact one of their tilted longitudinal faces, while taking care to place their small base inside said body.

19. Container according to claim 17, further comprising a leaktight seal, compatible with wine in spaces between, on the one hand, said peripheral grooves and front and rear peripheral edges of said body and, on the other hand, the tilted longitudinal faces.

20. Container according to claim 18, wherein the body of the external enclosure has a polygonal shape of which the inside cross-section is homothetic and of very slightly larger dimensions that those of the external cross-section of the body of the internal enclosure.

21. Container according to claim 14, further comprising an automatic wine-producing system.

22. Container according to claim 21, wherein said automatic wine-producing system comprises at least:
one wine oxygenating device,
one independent thermoregulating device,
one pressure control device in the internal enclosure, and
one graphic management interface, enabling all devices of the automatic wine-producing system to be managed, and to quickly and simply control the progression of the wine's fermentation state.

23. Container according to claim 22, wherein said oxygenation device comprises an oxygen consumption measuring body, a wine oxygen saturation control body and an oxygen inflow body.

24. Container according to claim 22, wherein said independent thermoregulating device comprises a wine temperature measuring probe and a heat exchanger, connected to a network of pipes, positioned over the whole of the external periphery of the body of the external enclosure, said pipes heating or cooling through conduction of said external enclosure.

25. Container according to claim 21, wherein said automatic wine-producing system comprises a brewing device and a sediment-extracting device.

26. Method of cleaning and sterilising the container according to claim 14 in view of its storage when empty, comprising the following steps:
brushing the whole of the inside surface of the internal enclosure,
cleaning the whole of the inside surface of the internal enclosure with a high-pressure water jet,
deep-sterilising the inside of the internal enclosure with steam or with ultraviolet rays.
